(19) United States
(12) Patent Application Publication
(10) Pub. No.: US 2016/0287583 A1
(43) Pub. Date: Oct. 6, 2016

(54) ORAL DISPERSIBLE COMPOSITION OF A DPP-IV INHIBITOR

(71) Applicant: RANBAXY LABORATORIES LIMITED, New Delhi (IN)

(72) Inventors: Vinod Kumar ARORA, Gurgaon (IN); Rathinasabapathy VENKATESHWARAN, Madurai (IN); Sumit MADAN, New Delhi (IN)

(73) Assignee: Ranbaxy Laboratories Limited, Delhi (IN)

(21) Appl. No.: 15/037,381

(22) PCT Filed: Nov. 13, 2014
(86) PCT No.: PCT/IB2014/066025

§ 371 Date: May 18, 2016

(30) Foreign Application Priority Data
Nov. 18, 2013 (IN) 3376/DEL/2013

(51) Int. Cl. A61K 31/4985; A61K 45/06; A61K 9/20; A61K 9/00; A61K 47/48

(52) U.S. Cl. CPC A61K 31/4985; A61K 9/0056; A61K 47/48969; A61K 9/2018; A61K 9/2095; A61K 45/06; A61K 9/2013; A61K 9/2054; A61K 9/2027

(57) ABSTRACT

The present invention relates to oral dispersible compositions comprising a DPP-IV inhibitor and processes for their preparation. It further relates to a method of treating diabetes by administering said oral dispersible compositions.

### FIELD OF THE INVENTION

The present invention relates to oral dispersible compositions comprising a DPP-IV inhibitor and processes for their preparation. It further relates to a method of treating diabetes by administering said oral dispersible compositions.

### BACKGROUND OF THE INVENTION

DPP-IV (dipeptidyl peptidase IV) is an enzyme that catalyzes the conversion of glucagon like peptide-1 (GLP-1) from its active form to its inactive form. DPP-IV inhibitors, also commonly known as gliptins, competitively inhibit the enzyme DPP-IV, thereby increasing the endogenous concentration of GLP-1, which further augments insulin secretion and improves the glycemic profile of patients with diabetes.

Presently, DPP-IV inhibitors such as sitagliptin, vildagliptin, saxagliptin, teneligliptin, alogliptin, and linagliptin are available as conventional tablet dosage forms. Although conventional tablet dosage forms constitute a preferred route of administration, certain groups of patients including geriatric, bed-ridden, uncooperative, nauseated, on reduced water intake, and patients having dysphagia, i.e., difficulty in swallowing, face problems while taking these dosage forms. Moreover, patients while travelling may have little or no access to water, limiting the use of conventional tablet dosage forms. As antidiabetic drugs are prescribed chronically, such a problem could lead to a high level of patient non-compliance. In view of this, oral dispersible compositions provide the best alternative over conventional tablet dosage forms. Oral dispersible compositions which rapidly disintegrate on contact with saliva or in a small amount of water, offer increased convenience and ease of administration with the potential to achieve better patient compliance.

Further, as DPP-IV inhibitors are generally bitter in taste, conventional tablet dosage forms are available as film-coated tablets in which the outer film coating is used for taste-masking. Therefore, there is no means of adjusting the dose, as film-coated tablets are difficult to break. The present invention provides a significant advance over the available conventional film-coated tablet dosage forms of DPP-IV inhibitors, as oral dispersible compositions of the present invention are uncoated and, further, can be a scored or divisible tablet having a scored line on the surface of the tablet. The scored line enables the patient to easily divide the tablet by applying minimal force in order to adjust the dose.

As oral dispersible compositions are designed to rapidly disintegrate as the drug comes in direct contact with the tongue, it remains a challenge to the formulators to effectively mask the taste of bitter drugs such as DPP-IV inhibitors to increase the acceptability for these compositions.

Hence, there exists a need in the art for oral dispersible compositions of DPP-IV inhibitors with an acceptable taste. The present invention teaches oral dispersible compositions of a DPP-IV inhibitor with an acceptable taste. These compositions exhibit enhanced structural integrity and further ensure dosage uniformity by forming a homogeneous dispersion.

### SUMMARY OF THE INVENTION

The present invention provides oral dispersible compositions of a DPP-IV inhibitor that will rapidly disintegrate within three minutes. These compositions have an acceptable taste, offer a pleasant mouth feel, and leave minimal residue in the mouth after administration. The present invention further provides processes for preparing said oral dispersible compositions. It also provides a method of treating diabetes by administering said oral dispersible compositions.

### DETAILED DESCRIPTION OF THE INVENTION

A first aspect of the present invention provides an oral dispersible composition comprising a DPP-IV inhibitor and one or more pharmaceutically acceptable excipients, wherein the composition disintegrates within three minutes.

According to one embodiment of this aspect, there is provided an oral dispersible composition comprising a DPP-IV inhibitor and one or more pharmaceutically acceptable excipients, wherein the DPP-IV inhibitor is coated with a polymer selected from pH-independent polymers or pH-dependent polymers.

According to another embodiment of this aspect, there is provided an oral dispersible composition comprising a DPP-IV inhibitor and one or more pharmaceutically acceptable excipients, wherein the DPP-IV inhibitor is complexed with a complexing agent selected from a cyclodextrin or an ion-exchange resin.

According to another embodiment of this aspect, there is provided an oral dispersible composition comprising a DPP-IV inhibitor and one or more pharmaceutically acceptable excipients, wherein the pharmaceutically acceptable excipients are selected from the group comprising sweeteners, disintegrants, fillers, suspending agents, lubricants, binders, wetting agents, coloring agents, flavoring agents, and combinations thereof.

According to another embodiment of this aspect, there is provided an oral dispersible composition comprising a DPP-IV inhibitor and one or more pharmaceutically acceptable excipients, wherein the sweeteners are selected from the group consisting of a sugar alcohol, a non-nutritive sugar based sweetener, and combinations thereof.

According to another embodiment of this aspect, there is provided an oral dispersible composition comprising a DPP-IV inhibitor and one or more pharmaceutically acceptable excipients, wherein the composition is stable.

According to another embodiment of this aspect, there is provided an oral dispersible composition comprising a DPP-IV inhibitor and one or more pharmaceutically acceptable excipients, wherein the composition further comprises one or more additional antidiabetic drugs.

A second aspect of the present invention provides a process for preparing an oral dispersible composition comprising a DPP-IV inhibitor comprising:
(i) blending a DPP-IV inhibitor with one or more pharmaceutically acceptable excipients;
(ii) optionally granulating the blend of step (i) with a suitable solvent; and
(iii) compressing the blend of step (i) or the granules of step (ii) into a tablet using appropriate tooling.

A third aspect of the present invention provides a process for preparing an oral dispersible composition comprising a DPP-IV inhibitor, comprising:
(i) blending a DPP-IV inhibitor and a polymer;
(ii) granulating the blend of step (i) using a suitable solvent;
(iii) mixing the granules of step (ii) with one or more pharmaceutically acceptable excipients; and
(iv) compressing the blend of step (iii) into a tablet using appropriate tooling.

A fourth aspect of the present invention provides a process for preparing an oral dispersible composition comprising a DPP-IV inhibitor, comprising:
(i) dissolving or dispersing a DPP-IV inhibitor and a polymer in a suitable solvent;
(ii) removing the solvent from the solution or dispersion of step (i) using a suitable solvent evaporation technique to obtain the dry material;
(iii) mixing the dry material of step (ii) with one or more pharmaceutically acceptable excipients; and
(iv) compressing the mixture of step (iii) into a tablet using an appropriate tooling.

A fifth aspect of the present invention provides a process for preparing an oral dispersible composition comprising a DPP-IV inhibitor, comprising:
(i) dissolving or dispersing a DPP-IV inhibitor and a complexing agent or an ion-exchange resin in a suitable solvent;
(ii) removing the solvent from the solution or dispersion of step (i) using a suitable solvent evaporation technique to obtain the dry material;
(iii) mixing the dry material of step (ii) with one or more pharmaceutically acceptable excipients; and
(iv) compressing the mixture of step (iii) into a tablet using appropriate tooling.

A sixth aspect of the present invention provides a method of treating diabetes by administering an oral dispersible composition comprising a DPP-IV inhibitor and one or more pharmaceutically acceptable excipients, wherein the composition disintegrates within three minutes.

According to one embodiment of this aspect, there is provided a method of treating diabetes by administering an oral dispersible composition comprising a DPP-IV inhibitor and one or more pharmaceutically acceptable excipients, wherein the method comprises sequential or simultaneous administration of one or more additional antidiabetic drugs.

The term “dispersible,” as used herein, is intended for dosage forms that completely disperse in water in a short period of time, i.e., less than about three minutes, to form a solution, a non-gritty suspension, or a slurry when placed either in water or in the oral cavity.

The term “homogeneous dispersion,” as used herein, means that the dispersion produced upon contact with water or saliva ensures the uniformity of the drug content for a reasonable period of time.

The term “stable,” as used herein, refers to chemical stability, wherein not more than 5% w/w of total related substances are formed on storage at 40° C. and 75% relative humidity or at 25° C. and 60% relative humidity for a period of at least three months to the extent necessary for the sale and use of the composition.

The term “pH-independent polymer,” as used herein, means any polymer that dissolves/disperses independent of pH. Suitable examples of pH-independent polymers are selected from the group consisting of cellulose derivatives such as ethyl cellulose, hydroxypropyl methyl cellulose, hydroxypropyl cellulose, hydroxypropyl ethyl cellulose, hydroxyethyl cellulose, and methylcellulose; gums such as xanthan gum; acrylate polymers such as of Eudragit® RL 30 D, Eudragit® RS 30 D; polyethylene oxide; and combinations thereof.

The term “pH-dependent polymer,” as used herein, means any polymer that dissolves/disperses at a specified pH, preferably at an acidic pH in the stomach, but are resistant to the near neutral pH in the mouth. Suitable examples of pH-dependent polymers are selected from the group consisting of dimethyl aminoethyl methacrylate copolymers such as Eudragit® E PO; cellulose esters and their derivatives such as hydroxypropyl methyl cellulose acetate succinate; and combinations thereof. These polymers may be used as a dry powder or as a dispersion or solution in a suitable solvent.

The term “cyclodextrin,” as used herein, refers to an agent capable of forming an inclusion complex and masking the bitter taste of a DPP-IV inhibitor by either decreasing its oral solubility on ingestion or decreasing the amount of DPP-IV inhibitor particles exposed to taste buds, thereby reducing the perception of bitter taste. Suitable cyclodextrins are selected from the group consisting of alpha cyclodextrin, gamma cyclodextrin, beta cyclodextrin, cyclodextrin derivative, and combinations thereof. A preferred cyclodextrin of the present invention is beta cyclodextrin. For optimal taste-masking, DPP-IV inhibitor to cyclodextrin weight ratio may be varied from 1:0.1 to 0.1:1.

The term “ion-exchange resin,” as used herein, refers to an agent which can exchange its mobile ions of equal charge with the surrounding medium. Ion-exchange resins are high molecular weight water-insoluble polyelectrolytes, and therefore are not absorbed by the body and remain inert. Examples of suitable ion-exchange resins are Amberlite® CG50, Amberlite® IRP64, Amberlite® IRP88, Amberlite® IRP69, Indion™ 204, Indion™ 214, Indion™ 234, Indion™ 244, Indion™ 254, and combinations thereof. The preferred ion-exchange resin is Amberlite® IRP64. For optimal taste-masking, the DPP-IV inhibitor to ion-exchange resin weight ratio may be varied from 1:0.1 to 0.1:1.

The term “DPP-IV inhibitors,” as used herein, refers to the class of drugs that exhibits inhibition of the DPP-IV enzyme. Suitable examples of DPP-IV inhibitors are selected from the group comprising sitagliptin, vildagliptin, saxagliptin, teneligliptin, alogliptin, linagliptin, and pharmaceutically acceptable salts or esters thereof. The pharmaceutically acceptable salts or esters may be prepared from an inorganic acid or an organic acid selected from the group comprising hydrochloric acid, hydrobromic acid, hydroiodic acid, nitric acid, carbonic acid, bicarbonic acid, sulfuric acid, phosphoric acid, bisulphonic acid, oxalic acid, formic acid, acetic acid, propionic acid, succinic acid, glycolic acid, gluconic acid, lactic acid, malic acid, tartaric acid, citric acid, ascorbic acid, glucuronic acid, maleic acid, fumaric acid, pyruvic acid, aspartic acid, glutamic acid, benzoic acid, anthranilic acid, mesylic acid, salicyclic acid, p-hydroxybenzoic acid, phenylacetic acid, mandelic acid, embonic acid, methanesulfonic acid, ethanesulfonic acid, benzenesulfonic acid, pantothenic acid, 2-hydroxyethanesulfonic acid, toluenesulfonic acid, sulfanilic acid, cyclohexyl aminosulfonic acid, stearic acid, alginic acid, galactaric acid, and galacturonic acid. DPP-IV inhibitors as used in the composition of the present invention may be present as crystalline, amorphous, anhydrous, hydrous, solvates, prodrugs, chelates, and complex forms. The dose of any of the DPP-IV inhibitors depends upon the individual drug used in the oral dispersible composition of the present invention.

Suitable additional antidiabetic drugs are selected from the group comprising acarbose, miglitol, repaglinide, nateglinide, glibenclamide, gliclazide, glimepiride, glipizide, tolbutamide, metformin, phenformin, rosiglitazone, pioglitazone, troglitazone, faraglitazar, englitazone, darglitazone, isaglitazone, reglitazar, rivoglitazone, liraglutide, muraglitazar, peliglitazar, tesaglitazar, canagliflozin, dapagliflozin, remogliflozin, sergliflozin, and pharmaceutically acceptable salts or esters thereof.

The term “composition,” as used herein, refers to tablets, pellets, pills, granules, and powders. Pellets, pills, granules, and powders in the form of a simple mixture can be filled into sachets that can be emptied into water. Preferably, the composition of the present invention is a tablet.

The oral dispersible compositions of the present invention are intended to be dispersed in water prior to administration, resulting in a homogenous dispersion. Further, these can also be kept in the mouth to form a dispersion in saliva.

The dispersible composition of the present invention comprises pharmaceutically acceptable excipients selected from the group consisting of sweeteners, disintegrants, fillers, suspending agents, lubricants, binders, wetting agents, coloring agents, flavoring agents, or combinations thereof.

The sweetener used in the present invention is selected from the group consisting of a sugar alcohol, a non-nutritive sugar based sweetener, and combinations thereof.

Suitable sugar alcohols are selected from the group consisting of erythritol, theritol, ribitol, arabinitol, xylitol, allitol, dulcitol, glucitol, sorbitol, mannitol, altritol, iditol, maltitol, lactitol, isomalt, and combinations thereof.

Non-nutritive sugar based sweeteners are sugar substitutes which have a sweet taste but are non-caloric. Suitable non-nutritive sugar based sweeteners are selected from the group consisting of aspartame, alitame, acesulfame-K, cyclamate, stevioside, glycyrrhizin, sucralose, neohesperidin, dihydrochalcone, thaumatin, sodium saccharin, and combinations thereof.

Suitable disintegrants are selected from the group comprising croscarmellose sodium; low-substituted hydroxypropylcellulose (L-HPC); co-processed mannitol; sodium starch glycolate; carboxymethyl cellulose; calcium carboxymethyl cellulose; cross-linked polyvinyl pyrrolidone; natural, modified, or pregelatinized starch; microcrystalline cellulose; gums; alginic acid; and combinations thereof.

Suitable fillers are selected from the group comprising microcrystalline cellulose; calcium disulfate; calcium trisulfate; calcium carbonate; calcium phosphate dibasic; calcium phosphate tribasic; kaolin; calcium silicate; maltodextrin; sugar alcohols such as xylitol, erythritol, sorbitol, and mannitol; microcrystalline cellulose; and combinations thereof.

Suitable suspending agents are selected from the group comprising water-dispersible celluloses, propylene glycol, polyethylene glycol, glycerin, or mixtures thereof. In particular, water-dispersible celluloses are co-processed spray dried forms of microcrystalline cellulose and carboxymethyl cellulose sodium. These have been marketed under trade names such as Avicel® RC-501 (containing 7.1% to 11.9% of sodium carboxymethyl cellulose) and Avicel® RC-581 (containing 8.3% to 13.8% of sodium carboxymethyl cellulose).

Suitable lubricants are selected from the group comprising magnesium stearate, colloidal silicon dioxide, stearic acid, calcium stearate, zinc stearate, sodium stearyl fumarate, talc, microcrystalline wax, silica gel, and combinations thereof.

Suitable binders are selected from the group comprising methylcellulose, hydroxypropyl cellulose, hydroxypropyl methyl cellulose, polyvinylpyrrolidone, gelatin, gum arabic, ethyl cellulose, polyvinyl alcohol, microcrystalline cellulose, pullulan, pregelatinized starch, agar, tragacanth, sodium alginate, propylene glycol, carboxyvinyl polymers, and combinations thereof.

Suitable wetting agents are selected from the group consisting of sodium lauryl sulphate, sorbitan esters of fatty acids, sorbitan monolaurate, sorbitan monooleate, sorbitan trioleate, sorbitan monopalmitate, sorbitan monostearate, sorbitan tristearate, ethylene oxide-propylene oxide block copolymers, lecithins, oleic acid and oleic acid salts, propylene glycol monostearate and monolaurate, glycerol monostearate and monooleate, fatty alcohol-polyethylene glycol ethers, fatty acid-polyethylene glycol esters, sodium dodecyl sulphate, dioctyl sodium sulphosuccinate, ethoxylated mono- and di-glycerides, sucrose fatty acid esters, fatty acid salts, ethoxylated triglycerides, polyoxyethylated hydrogenated castor oil, sterol, and combinations thereof.

Suitable coloring agents are selected from the group consisting of FDA approved coloring agents; natural coloring agents; natural juice concentrates; pigments such as titanium dioxide, iron oxide, and zinc oxide; and combinations thereof.

Suitable flavoring agents are selected from the group consisting of FDA approved flavoring agents and natural flavoring agents. Such flavors may include peppermint, grapefruit, orange, lime, lemon, mandarin, pineapple, strawberry, raspberry, mango, passion fruit, kiwi, apple, pear, peach, apricot, cherry, grape, banana, cranberry, blueberry, black currant, red currant, gooseberry, lingon berries, cumin, thyme, basil, camille, valerian, fennel, parsley, chamomile, tarragon, lavender, dill, bargamot, salvia, aloe vera balsam, spearmint, eucalyptus, and combinations thereof.

The oral dispersible compositions of the present invention can be prepared by conventional techniques such as direct compression, dry granulation, or wet granulation.

Suitable solvents used for granulation or for forming a solution or dispersion as described above are selected from the group consisting of water, ethanol, methylene chloride, isopropyl alcohol, acetone, methanol, and combinations thereof.

Solvent evaporation techniques include techniques well known in the art, such as air drying, vacuum drying, or heating.

The following examples represent various embodiments according to the present invention. The examples are given solely for the purposes of illustration and are not to be construed as limitations of the present invention, as many variations thereof are possible without departing from the spirit and scope of the invention.

### EXAMPLES

### Example 1

| Ingredients | Quantity (% w/w) |
|---|---|
| Sitagliptin phosphate monohydrate | 15.67 |
| Eudragit ® E PO | 5.55 |
| Co-processed mannitol | 37.32 |
| Sucralose | 9.15 |
| Xylitol | 15.24 |
| Hydroxypropyl methyl cellulose | 6.09 |
| Croscarmellose sodium | 7.32 |
| Peppermint flavor | 2.44 |
| Magnesium stearate | 1.22 |
| Purified water | q.s. |

### Procedure:

1. Sitagliptin phosphate monohydrate and Eudragit® E PO were blended together.
2. The blend of step 1 was granulated using purified water to obtain granules.
3. The granules of step 2 were dried using a fluidized bed dryer.
4. Co-processed mannitol, sucralose, xylitol, hydroxypropyl methyl cellulose, and croscarmellose sodium were mixed together.
5. The granules of step 3 were blended with the mixture of step 4.
6. Peppermint flavor and magnesium stearate were added to the blend of step 5.
7. The mixture of step 6 was compressed into a tablet dosage form using appropriate tooling.

### Example 2

| Ingredients | Quantity (% w/w) |
|---|---|
| Sitagliptin phosphate monohydrate | 15.67 |
| Beta cyclodextrin | 7.84 |
| Co-processed mannitol | 35.03 |
| Sucralose | 9.15 |
| Xylitol | 15.24 |
| Hydroxypropyl methyl cellulose | 6.09 |
| Croscarmellose sodium | 7.32 |
| Peppermint flavor | 2.44 |
| Magnesium stearate | 1.22 |
| Purified water | q.s. |

### Procedure:

1. Beta cyclodextrin was dissolved in a suitable quantity of purified water.
2. Sitagliptin phosphate monohydrate was slowly added into the solution of step 1.
3. The solution of step 2 was dried under vacuum.
4. Co-processed mannitol, sucralose, xylitol, hydroxypropyl methyl cellulose, and croscarmellose sodium were mixed together.
5. The dried material of step 3 was blended with the mixture of step 4.
6. Peppermint flavor and magnesium stearate were added to the blend of step 5.
7. The mixture of step 6 was compressed into a tablet dosage form using appropriate tooling.

### Example 3

| Ingredients | Quantity (% w/w) |
|---|---|
| Sitagliptin phosphate monohydrate | 16.06 |
| Amberlite ® IRP64 | 5.63 |
| Co-processed mannitol | 35.81 |
| Sucralose | 9.37 |
| Xylitol | 15.63 |
| Hydroxypropyl methyl cellulose | 6.25 |
| Croscarmellose sodium | 7.50 |
| Peppermint flavor | 2.50 |
| Magnesium stearate | 1.25 |
| Purified water | q.s. |

### Procedure:

1. Amberlite® IRP64 was dispersed in a suitable quantity of purified water.
2. Sitagliptin phosphate monohydrate was slowly added into the dispersion of step 1.
3. The solution of step 2 was dried under vacuum.
4. Co-processed mannitol, sucralose, xylitol, hydroxypropyl methyl cellulose, and croscarmellose sodium were mixed together.
5. The dried material of step 3 was blended with the mixture of step 4.
6. Peppermint flavor and magnesium stearate were added to the blend of step 5.
7. The mixture of step 6 was compressed into a tablet dosage form using appropriate tooling.

### Example 4

| Ingredients | Quantity (% w/w) |
|---|---|
| Sitagliptin phosphate monohydrate | 16.47 |
| Ethyl cellulose | 3.21 |
| Co-processed mannitol | 36.73 |
| Sucralose | 9.62 |
| Xylitol | 16.03 |
| Hydroxypropyl methyl cellulose | 6.41 |
| Croscarmellose sodium | 7.69 |
| Peppermint flavor | 2.56 |
| Magnesium stearate | 1.28 |
| Ethanol | q.s. |

### Procedure:

1. Ethyl cellulose was dissolved in a suitable quantity of ethanol
2. Sitagliptin phosphate monohydrate was slowly added into the solution of step 1.
3. The solution of step 2 was dried by heating.
4. Co-processed mannitol, sucralose, xylitol, hydroxypropyl methyl cellulose, and croscarmellose sodium were mixed together.
5. The dried material of step 3 was blended with the mixture of step 4.
6. Peppermint flavor and magnesium stearate were added to the blend of step 5.
7. The mixture of step 6 was compressed into a tablet dosage form using appropriate tooling.

### Example 5

| Ingredients | Quantity (% w/w) |
|---|---|
| Sitagliptin phosphate monohydrate | 16.58 |
| Co-processed mannitol | 39.55 |
| Sucralose | 9.68 |
| Xylitol | 16.13 |
| Hydroxypropyl methyl cellulose | 6.45 |
| Croscarmellose sodium | 7.74 |
| Peppermint flavor | 2.58 |
| Magnesium stearate | 1.29 |

### Procedure:

1. Sitagliptin phosphate monohydrate, co-processed mannitol, sucralose, xylitol, hydroxypropyl methyl cellulose, and croscarmellose sodium were mixed together.
2. Peppermint flavor and magnesium stearate were blended with the mixture of step 1.
3. The mixture of step 2 was compressed into a tablet dosage form using appropriate tooling.

### Disintegration Data

Three tablets prepared according to Example 1 were tested using British Pharmacopoeia disintegration apparatus. The results of the disintegration tests are presented in Table 1.

TABLE 1
Disintegration Times

| Example 1 | Disintegration Time |
|---|---|
| Tablet 1 | 1 minute 48 seconds |
| Tablet 2 | 2 minutes 03 seconds |
| Tablet 3 | 2 minutes 20 seconds |

1. An oral dispersible composition comprising a DPP-IV inhibitor and one or more pharmaceutically acceptable excipients, wherein the composition disintegrates within three minutes.

2. The oral dispersible composition according to claim 1, wherein the DPP-IV inhibitor is coated with a polymer selected from a pH-independent polymer or a pH-dependent polymer.

3. The oral dispersible composition according to claim 2, wherein the pH-independent polymer is selected from the group consisting of cellulose derivatives, gums, acrylate polymers, polyethylene oxide, and combinations thereof.

4. The oral dispersible composition according to claim 2, wherein the pH-dependent polymer is selected from the group consisting of dimethyl aminoethyl methacrylate copolymer, cellulose esters and their derivatives, and combinations thereof.

5. The oral dispersible composition according to claim 1, wherein the DPP-IV inhibitor is complexed with a complexing agent selected from a cyclodextrin or an ion-exchange resin.

6. The oral dispersible composition according to claim 5, wherein the cyclodextrin is selected from the group consisting of alpha cyclodextrin, gamma cyclodextrin, beta cyclodextrin, cyclodextrin derivatives, or combinations thereof.

7. The oral dispersible composition according to claim 5, wherein the ion-exchange resin is selected from the group consisting of Amberlite® CG50, Amberlite® IRP64, Amberlite® IRP88, Amberlite® IRP69, Indion™ 204, Indion™ 214, Indion™ 234, Indion™ 244, Indion™ 254, and combinations thereof.

8. The oral dispersible composition according to claim 1, wherein the pharmaceutically acceptable excipient is selected from the group comprising sweeteners, disintegrants, fillers, suspending agents, lubricants, binders, wetting agents, coloring agents, flavoring agents, and combinations thereof.

9. The oral dispersible composition according to claim 8, wherein the sweetener is selected from the group consisting of a sugar alcohol, a non-nutritive sugar based sweetener, and combinations thereof.

10. The oral dispersible composition according to claim 9, wherein the sugar alcohol is selected from the group consisting of erythritol, theritol, ribitol, arabinitol, xylitol, allitol, dulcitol, glucitol, sorbitol, mannitol, altritol, iditol, maltitol, lactitol, isomalt, and combinations thereof.

11. The oral dispersible composition according to claim 9, wherein the non-nutritive sugar based sweetener is selected from the group consisting of aspartame, alitame, acesulfame-K, cyclamate, stevioside, glycyrrhizin, sucralose, neohesperidin, dihydrochalcone, thaumatin, sodium saccharin, and combinations thereof.

12. The oral dispersible composition according to claim 1, wherein the composition is selected from the group consisting of tablets, pellets, pills, granules, and powders.

13. The oral dispersible composition according to claim 12, wherein the composition is a tablet.

14. A process of preparing an oral dispersible composition comprising a DPP-IV inhibitor, comprising:
(i) blending a DPP-IV inhibitor with one or more pharmaceutically acceptable excipients;
(ii) optionally granulating the blend of step (i) with a suitable solvent; and
(iii) compressing the blend of step (i) or the granules of step (ii) into a tablet using appropriate tooling.

15. A process of preparing an oral dispersible composition comprising a DPP-IV inhibitor, comprising:
(i) blending a DPP-IV inhibitor and a polymer;
(ii) granulating the blend of step (i) using a suitable solvent;
(iii) mixing the granules of step (ii) with one or more pharmaceutically acceptable excipients; and
(iv) compressing the blend of step (iii) into a tablet using appropriate tooling.

16. A process of preparing an oral dispersible composition comprising a DPP-IV inhibitor, comprising:
(i) dissolving or dispersing a DPP-IV inhibitor and a polymer in a suitable solvent;
(ii) removing the solvent from the solution or dispersion of step (i) using a suitable solvent evaporation technique to obtain the dry material;
(iii) mixing the dry material of step (ii) with one or more pharmaceutically acceptable excipients; and
(iv) compressing the mixture of step (iii) into a tablet using appropriate tooling.

17. A process of preparing an oral dispersible composition comprising a DPP-IV inhibitor, comprising:
(i) dissolving or dispersing a DPP-IV inhibitor and a complexing agent or an ion-exchange resin in a suitable solvent;
(ii) removing the solvent from the solution or dispersion of step (i) using a suitable solvent evaporation technique to obtain the dry material;
(iii) mixing the dry material of step (ii) with one or more pharmaceutically acceptable excipients; and
(iv) compressing the mixture of step (iii) into a tablet using appropriate tooling.

18. A method of treating diabetes by administering the oral dispersible composition according to claim 1.

19. The method of treating diabetes according to claim 18, wherein the method further comprises sequential or simultaneous administration of one or more additional antidiabetic drugs.
